(19) United States
(12) Patent Application Publication
(10) Pub. No.: US 2010/0100019 A1
(43) Pub. Date: Apr. 22, 2010

(54) ABDOMINAL SUPPORT

(76) Inventors: Charlene Chen, San Francisco, CA (US); Nam C. Quach, San Francisco, CA (US)

Correspondence Address: DERGOSITS & NOAH LLP, Suite 1450, Four Embarcadero Center, San Francisco, CA, 94111, US

(21) Appl. No.: 12/255,840

(22) Filed: Oct. 22, 2008

(51) Int. Cl. A61F 5/00; A61F 5/28; A61F 7/00

(52) U.S. Cl. 602/19; 602/5; 128/100.1; 607/108; 607/114

(57) ABSTRACT

A device for supporting the abdominal region of the user. A fabric band is sized to be worn surrounding the abdominal region which supports strategically placed hook and loop regions. A pair of segments of stretch material, also having hook and loop regions at least one of their ends are capable of being selectively and removably appended to the fabric band to apply compression or restriction to the user's abdomen. The device is particularly useful in helping one to restore abdominal muscle and control particularly after pregnancy.

### TECHNICAL FIELD

The present invention involves a device for supporting the abdominal region of a user. The device is particularly advantageous in restoring abdominal muscle and control particularly after pregnancy, an event which generally results in the stretching of abdominal muscularature and resultant extension.

### BACKGROUND OF THE INVENTION

There are a number of devices intended to encircle the waist and lower back of a user for a plethora of purposes. Some of these devices act as back supports for many people suffer from lower back pain as a result of scoliosis or weak back muscles which do not adequately support the lower spine. Other products, including girdles and undergarment foundations are employed for aesthetic reasons causing a slimming effect under a dress or pants. Yet further articles of this nature are offered as medical appliances for such widely divergent uses as hernia support apparatus and bandage retention mostly employed post surgically.

Virtually all of the appliances discussed above commonly employ a single and, in some instances, multiple layers of stretch fabric which cinches upon a user causing abdominal and lower back compression. Even when multiple stretchable fabric layers are employed, they are invariably used as nothing more than redundant co-fabric with the notion being that if a single layer of stretchable fabric is good, then multiple layers of the same fabric must be better. There are, however, certain inherent disadvantages in taking such an approach.

When multi-layer stretchable foundation garments are employed, a user often experiences constriction resulting in limited movement. This is particularly the case when such multi-layer stretchable garments are applied to the lower back of a user which caused undue restriction in bending and twisting motions.

In addition to the above, it has been determined that few if any stretchable foundation garments have been designed to assist in a woman's attempt to rebuild abdominal muscles and consequently reestablish a relative flat stomach and pre-pregnancy conformity after the birth of a child. Pregnancy is unique in its causation of stretch and oftentimes ruptured abdominal musculature, a state which many women never recover from. It is common place for a woman to reminisce about the shape she had before pregnancy resigning herself to the realization that her pre-pregnancy look would never be restored. Virtually all elasticized or stretchable foundation garments currently known are incapable of assisting in the rebuilding of abdominal musculature.

It is thus an object of the present invention to provide a multi-layered stretchable garment which is capable of providing abdominal support while overcoming the disadvantages recited above.

It is yet a further object of the present invention to provide a foundation garment which is able to restore a woman's pre-pregnancy shape after childbirth.

These and further advantages will be more readily apparent when considering the following disclosure and appended claims.

### SUMMARY OF THE INVENTION

The present invention is directed to a device for supporting the abdominal region of a user comprising a substantially rectangularly shaped fabric, at least a segment of which is a stretch material having first and second distal ends and a releasable closure for attaching said distal ends to one another to form a band of said fabric sized to encircle the abdominal region of a user. As a first embodiment, a first hook and loop panel is positioned to reside proximate the abdomen of a user when said device is worn and a second hook and loop panel positioned to reside proximate the back of a user when said device is worn. Two segments of stretch material are provided, each segment having first and second ends and hook and loop regions located proximate said first and second ends for removable attachment of said segments to said substantially rectangularly shaped fabric, wherein said hook and loop region on the first end of each of said segments is removably attached to said first hook and loop panel and said hook and loop region on the second end of each of said segments is removably attached to said second hook and loop panel, said segments being sized to stretch from their at-rest lengths when said device is worn causing restriction to the abdominal region of a user.

As a second embodiment, the segments can be permanently stitched to the band of fabric in each of their first extremities while, at a second extremity, a strip of hook and loop fastener is appended. A pouch is strategically appended to the device to drape over the abdominal region of a user which includes a hook and loop fastener region which is sized to receive hook and loop fastener strips of the substantially rectangularly shaped fabric. In use, the rectangularly shaped fabric is pulled about the abdominal and lower back regions of a user and brought into proximity by releaseably appending ends of the rectangularly shaped fabric to the hook and loop region associated with the pouch. The segments of stretch fabric are then pulled to connect to the rectangularly shaped fabric in the lower back region of the user. This adds for the compression to the abdominal region and enhances the therapeutic benefits provided by the present invention.

The device can also be configured as a continuous band or loop of fabric rather than as the above-described rectangularly shaped fabric. In addition, indicia can be applied to it to enable a user to properly position the above-noted segments onto the first hook and loop panels.

### BRIEF DESCRIPTION OF THE FIGURES

FIG. 1 is a front perspective view illustrating a first iteration of the present invention.

FIG. 2 is a preferred embodiment of the invention of FIG. 1 illustrating the inclusion of a pouch for containing a hot or cold pack as desired.

FIG. 3 is a front perspective view illustrating a second embodiment of the present invention.

FIG. 4 is yet a further preferred embodiment of the invention depicted in FIG. 3.

### DETAILED DESCRIPTION OF THE INVENTION

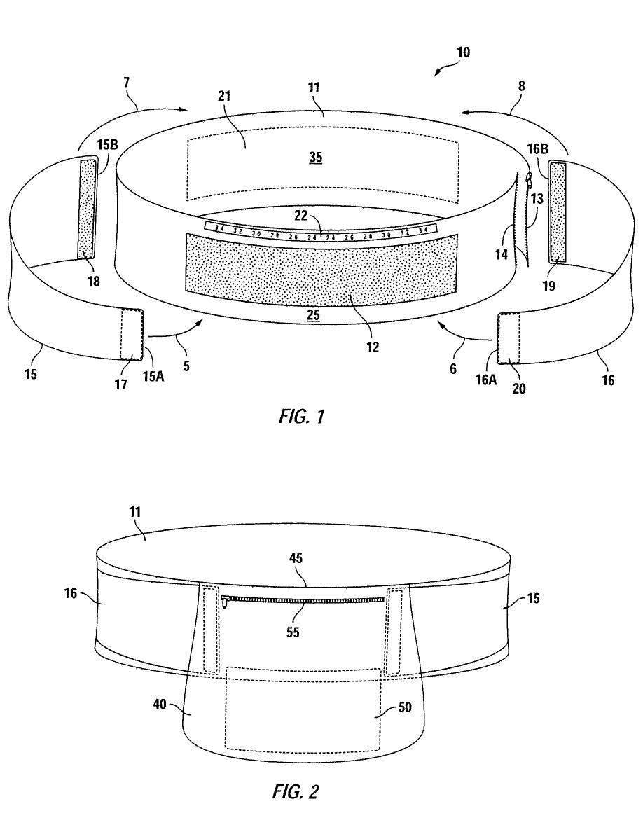

Turning first to FIG. 1, device 10 is depicted for supporting the abdominal region of a user. Device 10 can comprise a substantially rectangularly shaped material 11, at least a portion of which can be a stretch material such as, for example, neoprene or other lightweight stretchable fabric capable of returning to its original dimension repeatedly upon multiple use. Rectangularly-shaped material 11 is configured in the form of a band or a loop by attaching ends thereof by virtue of releasable closures 13 and 14. Such closures can be, for example, a member selected from the group consisting of zippers, hooks, snaps and hook and loop fasteners.

As an alternative to the illustration of FIG. 1, substantially rectangularly shaped fabric 11 can be constructed as a continuous band thus eliminating the need for releasable closures 13 and 14. The embodiment of FIG. 1 would perhaps be easier for a user to employ as fabric 11 can be applied to one's torso by simply wrapping the fabric around one's waist followed by connecting releasable closures 13 and 14. A continuous band on the other hand requires a user to slip the band over her feet and legs and lower torso much as one would slip into a girdle or similar support.

In continuing reference to FIG. 1, a first hook and loop panel 12 is positioned to reside in area 25 which would be proximate the abdomen of a user when the device is worn. A second hook and loop panel 21 is positioned in region 35 proximate the back of a user when said device is worn. Two segments of stretch material 15 and 16 are provided. The segments have first and second ends 15a-15b, 16a-16b, respectively, and hook and loop regions 17-18 and 19-20, respectively, for removable attachment of segments 15 and 16 to fabric 11 wherein hook and loop regions 17 and 20 on first ends 15a-16b are removably attached to first hook and loop panel 12 and hook and loop regions 18-19 on second ends 15b-16a of each of segments 15 and 16 are removably attached to second hook and loop panel 21. It is intended that when segments 15 and 16 are applied as shown schematically by arrows 5, 6, 7 and 8, the segments stretch from their at-rest lengths when the device is worn causing restriction to the abdominal region of a user. In other words, segments 15 and 16 are first applied by appending hook and loop regions 17 and 20 to hook and loop region 12 in the direction of arrows 5 and 6 whereupon second ends 15B and 16B of segments 15 and 16 are wrapped around fabric 11 and pulled in the direction of arrow 7 and 8 to cause compression when hook and loop regions 18 and 19 are appended to hook and loop region 21.

As a further preferred embodiment of the present invention, reference is once again made to FIG. 1. In order to assist a user in the appropriate positioning of segments 15 and 16 initially onto hook and loop region 12, waist size indicia 22 is applied to an edge of fabric 11 as shown. The indicia recites waist sizes to, again, suggest appropriate placement. As such, if a user's waist was 24 inches, ends 15A-16B of segments 15 and 16 would be placed quite close together upon their application to hook and loop region 12 while if a user's waist was 34 inches, ends 15A-16B would be applied further apart.

As yet a further embodiment of the present, reference is made to FIG. 2. In this instance, pouch 40 is provided by attachment to a region proximate top edge 45 of rectangularly shaped fabric or band 11. Pouch 40 is provided with opening 55 which can be selectively closable for receipt of a cold or hot pack 50. Cold and hot packs useful in application to pouch 40 are readily available and once inserted within pouch 40, said pouch drapes over the abdominal region of a user to apply heat or cold to the user's abdomen. This not only enhances a user's comfort but also can be therapeutic.

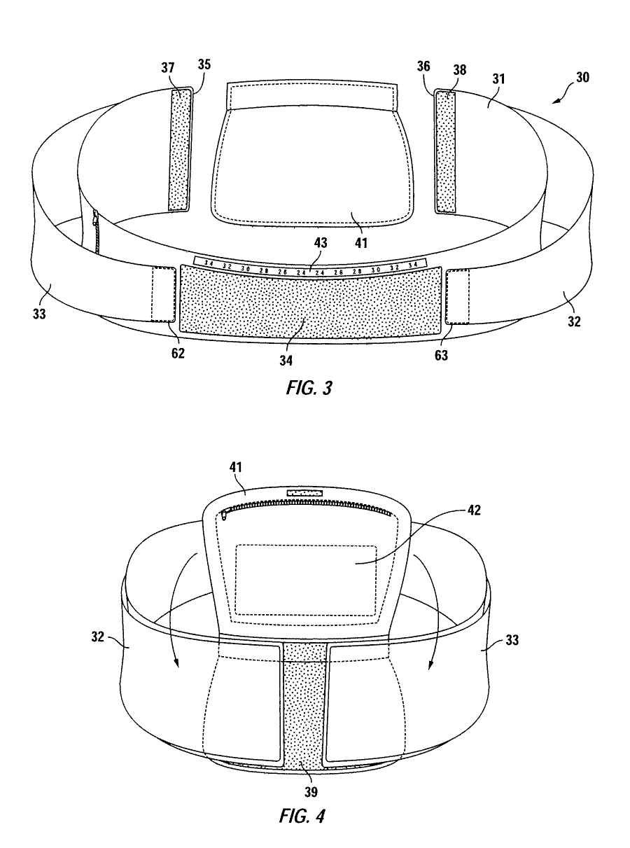

A second embodiment of the present invention is depicted in FIGS. 3 and 4. In this instance, substantially rectangularly shaped fabric 31, again constructed as either a continuous band or in releasable sections as shown. As noted in FIG. 3, band 31 ideally includes free ends 35 and 36, each of which supports a strip of hook and loop fasteners 37 and 38, respectively. Segments of stretch fabric 32 and 33 are sewn into seams proximate ends 35 and 36 while free ends 62 and 63 again support segments of hook and loop fasteners enabling free ends 62 and 63 to be selectively attached to hook and loop fastener region 34, the placement of which being dictated by waist size indicia 43 used as described with reference to element 22 of FIG. 1.

In applying the device of FIGS. 3 and 4 to the human torso, rectangularly shaped material 31 is wrapped around the user's waist and pouch 41, akin to pouch 40 of FIG. 2 is placed in the abdominal region of a user such that hook and loop strips 37 and 38 are caused to selectively attach to hook and loop region 39 located as part of the pouch 41 assembly. This enables hot or cold pack 42 to remain proximate the abdominal region for therapeutic purposes.

Once rectangularly shaped material 31 is wrapped about a user's abdomen and lower back, segments 32 and 33 are wrapped about the abdominal region for selective attachment to hook and loop region 34. Again, it is contemplated that segments 32 and 33 be made of a stretch fabric to create additional compression to the abdominal region beyond that offered simply by rectangularly shaped fabric 31. Sizing strip 43 can also be made part of this embodiment and used as strip 22 (FIG. 1). As is apparent from comparing the embodiments of FIGS. 1 and 3, sizing strips can be placed both on the front and back of the wrap, or on both sides while remaining with the teachings of the present invention.

What is claimed is:

1. A device for supporting the abdominal region of a user comprising a substantially rectangularly shaped fabric, at least a segment of which is stretch material having first and second distal ends and a releasable closure for attaching said distal ends to one another to form a band of said fabric sized to encircle the abdominal region of a user, a first hook and loop panel positioned to reside proximate the abdomen of a user when said device is worn and a second hook and loop panel positioned to reside proximate the back of a user when said device is worn, two segments of stretch material, each segment having first and second ends and hook and loop regions located proximate said first and second ends for removable attachment of said segments to said substantially rectangularly shaped fabric, wherein said hook and loop region on the first end of each of said segments is removably attached to said first hook and loop panel and said hook and loop region on the second end of each of said segments is removably attached to said second hook and loop panel, said segments being sized to stretch from their at-rest lengths when said device is worn causing restriction to the abdominal region of a user.

2. The device of claim 1 wherein said releasable closure comprises a member selected from the group consisting of zippers, hooks, snaps and hook and loop fasteners.

3. The device of claim 1 wherein waist size indicia is applied to said substantially rectangularly shaped fabric to enable a user to properly position said segments onto said first and second hook and loop panels.

4. The device of claim 1 further comprising a pouch located proximate the abdomen of a user when said device is worn.

5. The device of claim 4 wherein said pouch contains either a hot or cold pack.

6. A device for supporting the abdominal region of a user comprising a band of fabric, at least a segment of which is stretch material and sized to encircle the abdominal region of a user, a first hook and loop panel positioned to reside proximate the abdomen of a user when said device is worn and a second hook and loop panel positioned to reside proximate the back of a user when said device is worn, two segments of stretch material, each segment having first and second ends and hook and loop regions located proximate said first and second ends for removable attachment of said segments to said band, wherein said hook and loop region on the first end of each of said segments is removably attached to said first hook and loop panel and said hook and loop region on the second end of each of said segments is removably attached to said second hook and loop panel, said segments being sized to stretch from their at-rest lengths when said device is worn causing restriction to the abdominal region of a user.

7. The device of claim 6 wherein waist size indicia is applied to said substantially rectangularly shaped fabric to enable a user to properly position said segments onto said first and second hook and loop panels.

8. The device of claim 6 further comprising a pouch located proximate the abdomen of the user when said device is worn.

9. The device of claim 8 wherein said pouch contains either a hot or cold pack.

10. A device for supporting the abdominal region of a user comprising a substantially rectangularly shaped fabric, at least a segment of which is stretch material having first and second distal ends, each of which supports a hook and loop panel, and a field of a hook and loop panel located mid point of said distal ends, a pair of segments of stretch material, a first end of each segment being permanently affixed to said substantially rectangularly shaped fabric proximate said first and second distal ends, a second end of each segments supporting a hook and loop panel, each said hook and loop panel of said segments being releaseably attachable to said field of a hook and loop panel when said device is worn by a user, said substantially rectangularly shaped fabric being configured into a band for surrounding the abdominal region of a user by securing said distal ends to one another or to an intermediate hook and loop panel.

11. The device of claim 10 further comprising a pouch located proximate the abdomen of a user when said device is worn.

12. The device of claim 11 wherein said pouch is provided with said intermediate hook and loop panel.

13. The device of claim 11 wherein said pouch contains either a hot or cold pack.

14. The device of claim 10 wherein waist size indicia is applied to said substantially rectangularly shaped fabric to enable a user to properly position said segments onto said first and second hook and loop panels.
